(19) United States
(12) Patent Application Publication
(10) Pub. No.: US 2021/0106121 A1
(43) Pub. Date: Apr. 15, 2021

(54) LAMINATED SHEET, EXTERNAL PATCH, AND PACKAGING STRUCTURE

(71) Applicant: KANAE TECHNOS CO., LTD., Kanonji-shi (JP)

(72) Inventors: Tomokazu NAKANO, Kanonji-shi (JP); Mika KONDO, Kanonji-shi (JP); Ryoko FURUKAWA, Kanonji-shi (JP); Kosuke KATOH, Kanonji-shi (JP)

(73) Assignee: KANAE TECHNOS CO., LTD., Kanonji-shi (JP)

(21) Appl. No.: 16/823,343

(22) Filed: Mar. 19, 2020

(30) Foreign Application Priority Data
Oct. 15, 2019 (JP) 2019-188951

(51) Int. Cl. A45D 44/00; A45D 44/22

(52) U.S. Cl. CPC A45D 44/002; A61K 8/0212; A45D 44/22

(57) ABSTRACT

As a support for an external patch, a laminated sheet having a laminate structure of two or three layers in which a secondary sheet made of hydrophobic fibers is laminated on one side or both sides of a main sheet made of hydrophilic fibers is used.

### BACKGROUND

### Technical Field

The present invention relates to a laminated sheet having a laminated structure of two or three layers, an external patch using the laminated sheet as a support, and a packaging structure for the external patch.

### Related Art

A general external patch has a usage form of being distributed and stored in a state of being enclosed one by one or collectively in units of a plurality of sheets in a non-breathable bag material or container, that is, in a state of being packaged in a container and being opened when used. Of this type of external patch, the “face mask” used by being applied to the face is relatively large and is usually packaged in a container in a folded state.

However, many face masks are in the form of wet sheets in which a support such as a non-woven fabric is impregnated with lotion, etc., and the folded and overlapped parts stick together. Therefore, a complicated work of unfolding the sheets that stick together while separating these with fingers is required. Therefore, JP 2006-42981 A below proposes a folding structure of a face mask (face pack mask) that is easy to unfold.

### SUMMARY

However, even if the method of folding the support is devised, there are many cases where a user is unaware of the unfolding method assumed, and the unfolding operation is still difficult.

The present invention has been developed in view of the technical problem, and has an object to provide a novel laminated sheet that can be easily separated even when being wet and stick together, an external patch using the laminated sheet as a support, and a packaging structure for the external patch.

A laminated sheet of the present invention for solving the technical problem is a laminated sheet having a laminated structure of two or three layers in which a secondary sheet is laminated on one side or both sides of a main sheet, in which the main sheet is a nonwoven fabric made of hydrophilic fibers, and in which the secondary sheet is a nonwoven fabric made of hydrophobic fibers (hereinafter referred to as “present-invention laminated sheet”).

In the present-invention laminated sheet, a preferred mode is one in which the main sheet is made of hydrophilic fibers with an official regain of 7.0% or more, and in which the secondary sheet is made of hydrophobic fibers with an official regain of 5.5% or less.

In the present-invention laminated sheet, a preferred mode is one in which the main sheet is made of rayon fibers or cotton fibers, and in which the secondary sheet is made of PET fibers or polylactic acid fibers.

In the present-invention laminated sheet, a preferred mode is one in which a weight per unit area of the secondary sheet is larger than a weight per unit area of the main sheet.

In the present-invention laminated sheet, a preferred mode is one in which the main sheet has a weight per unit area of 10 to 50 g/m², and in which the secondary sheet has a weight per unit area of 15 to 60 g/m².

An external patch of the present invention for solving the technical problem is an external patch used by being applied to a skin surface including: a support including the present-invention laminated sheet; and a chemical solution that impregnates the support (hereinafter referred to as “present-invention external patch”).

A packaging structure of the present invention for solving the technical problem is a packaging structure in which the present-invention external patch is packaged in a container,

in which the support is enclosed in the container together with the chemical solution in a state of being folded (hereinafter referred to as “present-invention packaging structure”).

The present-invention laminated sheet can be easily separated even when being wet and stick together. Further, the external patch using the present-invention laminated sheet as a support, and the present-invention packaging structure in which the external patch is packaged in a container can be easily unfolded even when the support is folded.

### BRIEF DESCRIPTION OF DRAWINGS

FIG. 1A is a cross-sectional view showing an embodiment of a present-invention laminated sheet, and FIG. 1B is a front view showing the present-invention laminated sheet processed into a human face shape;

FIG. 2A is a perspective view showing an embodiment of a present-invention packaging structure, and FIG. 2B is a perspective view showing the present-invention packaging structure in a partially broken state so that a present-invention external patch present in a bag material can be visually recognized;

FIGS. 3A to 3E are perspective views sequentially showing steps of folding a support constituting the present-invention external patch;

FIG. 4A is a front view showing a state where the present-invention external patch is taken out from the present-invention packaging structure, and FIG. 4B is a perspective view showing a state of developing the present-invention external patch; and

FIG. 5 is a cross-sectional view showing another mode of the present-invention laminated sheet.

### DETAILED DESCRIPTION

Hereinafter, although embodiments of the present invention are described, the present invention is not limited to these embodiment.

### <Present-Invention Laminated Sheet 1>

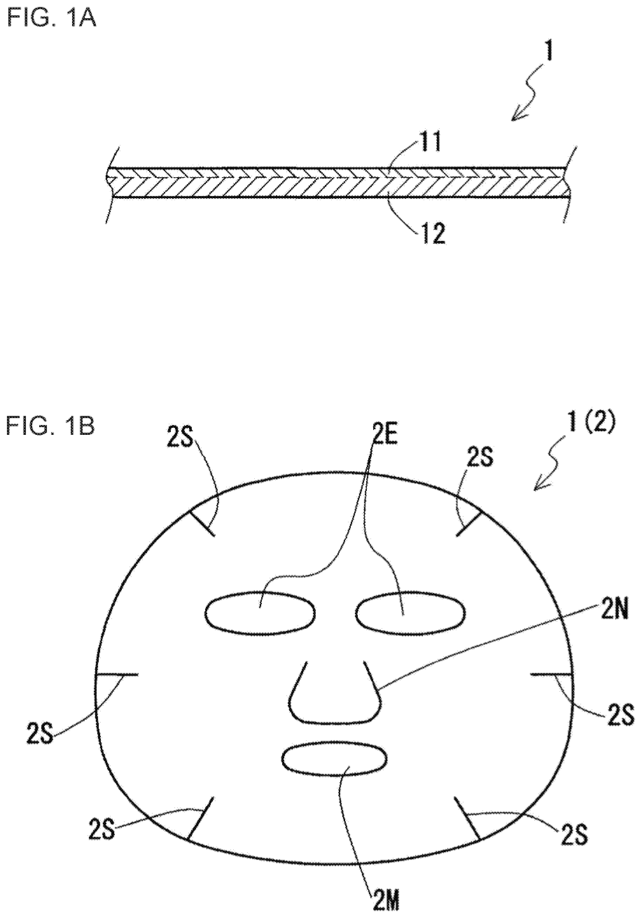

FIGS. 1A and 1B show an embodiment of a present-invention laminated sheet 1. As shown in FIG. 1A, the present-invention laminated sheet 1 has a two-layer laminated structure in which a “secondary sheet (12)” is laminated on one side of a “main sheet (11)”.

### —Main Sheet 11—

The main sheet 11 is a nonwoven fabric made of hydrophilic fibers. In the present embodiment, a cotton fiber (official regain: 8.5%) is selected as the hydrophilic fiber, and a nonwoven fabric (weight per unit area: 25 g/m²) in which this fiber is formed in a sheet shape is used as the main sheet 11.

### —Secondary Sheet 12—

The secondary sheet 12 is a nonwoven fabric made of hydrophobic fibers. In the present embodiment, a polylactic acid fiber (official regain: 0.5%) is selected as the hydrophobic fiber, and a nonwoven fabric (weight per unit area 30 g/m²) in which this fiber is formed into a sheet shape is used as the secondary sheet 12.

In the present embodiment, after the main sheet 11 and the secondary sheet 12 are stacked, a jet water flow is passed from one surface to the other surface, and the hydrophilic fiber and the hydrophobic fiber existing at the boundary are entwined, to thereby construct a laminated structure of the present-invention laminated sheet 1. Therefore, the boundary between the main sheet 11 and the secondary sheet 12 is not always clear.

In addition, as shown in FIG. 1B, in the present embodiment, the present-invention laminated sheet 1 is cut into a shape imitating a human face shape, and openings (2E, 2M) are provided in portions corresponding to the eyes and a portion corresponding to the mouth. In addition, a U-shaped cut 2N is provided in a portion corresponding to the nose and linear cuts 2S are provided at a plurality of locations on the outer edge so that the present-invention laminated sheet 1 can easily follow the three-dimensional shape of the face when being applied to the face.

### <Present-Invention External Patch 10 and Present-Invention Packaging Structure 100>

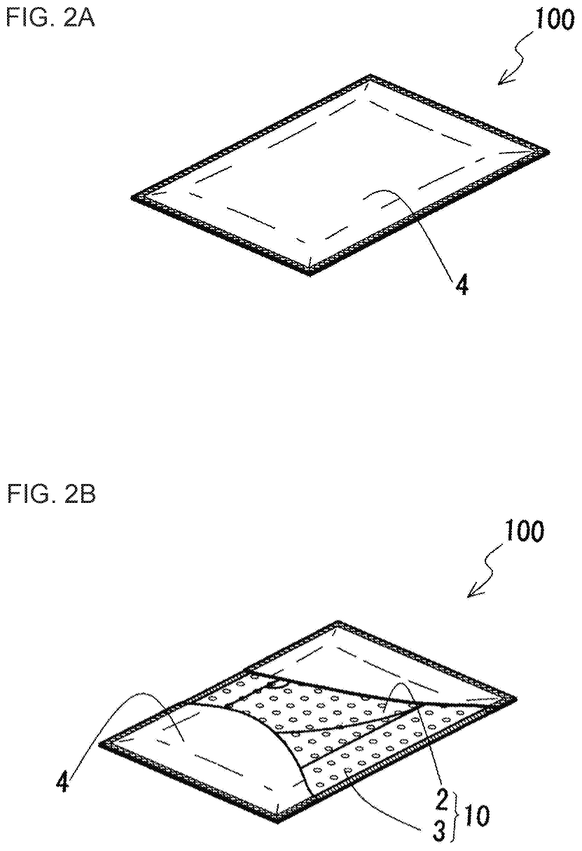

FIGS. 2A and 2B show an embodiment of a present-invention packaging structure 100 in which a present-invention external patch 10 is packaged in a container. The present-invention external patch 10 includes a “support (2)” and a “chemical solution (3)”. The present-invention packaging structure 100 has a structure in which the present-invention external patch 10 is enclosed in a “container (4)”.

### —Support 2—

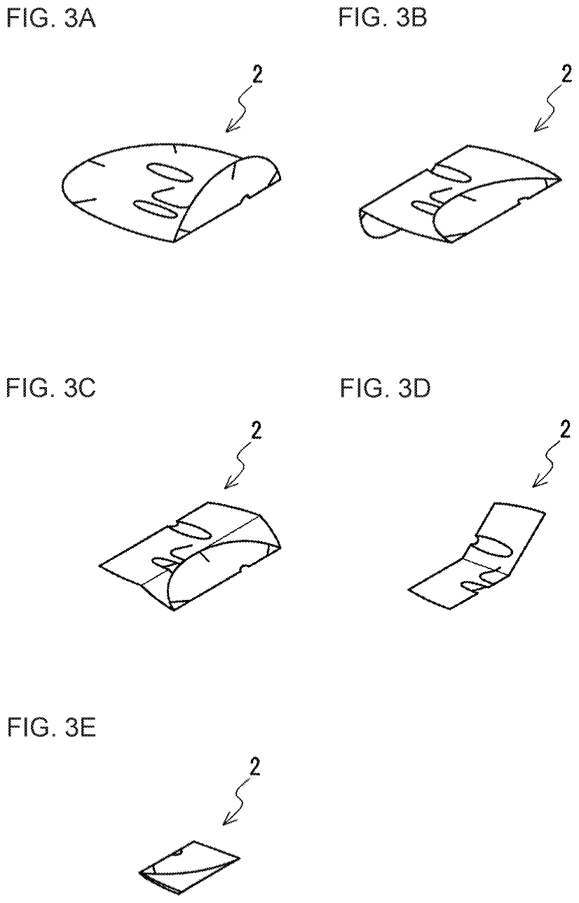

The support 2 is composed of the present-invention laminated sheet 1. In the present embodiment, as the support 2, the present-invention laminated sheet 1 cut into a shape imitating a human face shape is used (see FIG. 1B). In the present embodiment, the support 2 is folded through the steps shown in FIGS. 3A to 3E.

### —Chemical Solution 3—

The chemical solution 3 is supported by impregnating the support 2. In the present embodiment, lotion is used as the chemical solution 3.

### —Container 4—

The container 4 serves to enclose the support 2 in a folded state together with the chemical solution 3. In the present embodiment, a flat bag material having a four-side seal structure constructed by closing the periphery of two stacked rectangular non-breathable films (aluminum vapor deposition films) by heat sealing is used as the container 4.

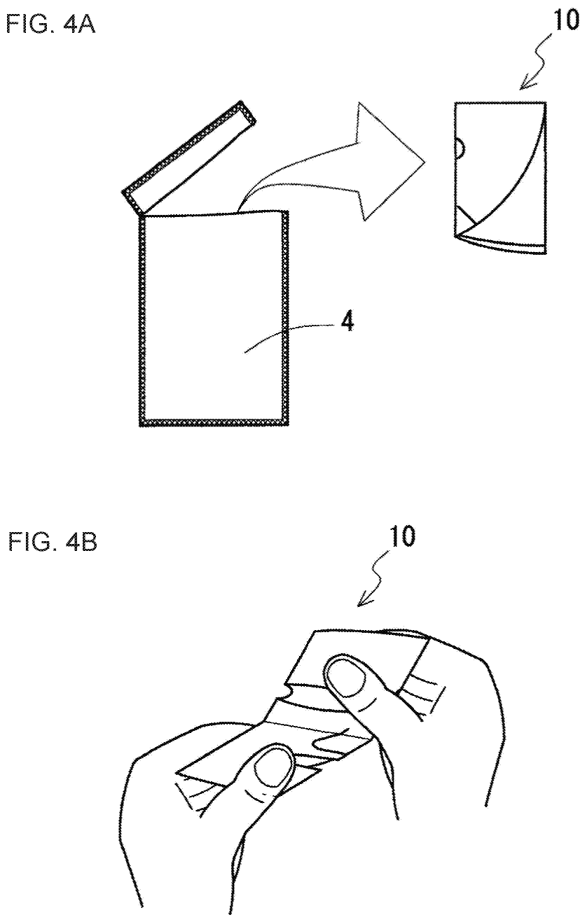

In using the present-invention external patch 10 present in the present-invention packaging structure 100, first, as shown in FIG. 4A, the container 4 is opened and the present-invention external patch 10 is taken out. Since the present-invention external patch 10 that has been taken out is in a folded state, an unfolding operation with fingers as shown in FIG. 4B is required.

When this unfolding operation is performed, the present-invention external patch 10 uses the present-invention laminated sheet 1 as the support 2. Therefore, even if the folded portions are wet and stuck together, the sheets are easily slipped in the part where the main sheet 11 and the secondary sheet 12 overlap with each other in a state of facing each other, and the part where the secondary sheet 12 overlaps with itself in a state of facing itself, so that the sheets are easily separated. As a result, the present-invention external patch 10 can be easily unfolded even when the support 2 is folded.

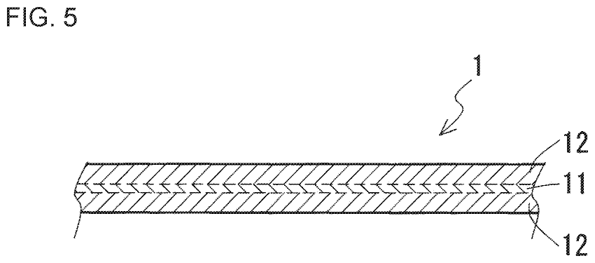

Incidentally, in the present embodiment, as the present present-invention laminated sheet 1, one having a two-layer laminated structure is used, but in the present invention, as the present-invention laminated sheet 1, one having a three-layer structure in which the secondary sheets 12 are laminated on both sides of the main sheet 11 as shown in FIG. 5 may be used. When the present-invention laminated sheet 1 having a three-layer laminated structure is used as the support 2 of the present-invention external patch 10 and is constructed as the present-invention packaging structure 100, there is no part where the main sheet 11 face itself in the folded portion, so that the present-invention external patch 10 can be unfolded even more easily.

In the present embodiment, cotton fibers are used as the hydrophilic fibers constituting the main sheet 11, and polylactic acid fibers are used as the hydrophobic fibers constituting the secondary sheet 12. However, the hydrophilic fibers and the hydrophobic fibers are not limited to these materials. It has been confirmed that the sheets can be easily separated even when being wet and stuck together by constructing the present-invention laminated sheet 1 by the main sheet 11 made of hydrophilic fibers having an official regain of 7.0% or more (more preferably 8.5% or more) and the secondary sheet 12 made of hydrophobic fibers having an official regain of 5.5% or less (more preferably 4.5% or less). In the present invention, “official regain” refers to a value measured according to JIS0105 “General rules for physical testing of textile products” and ISO139 (Textiles-Standard atmospheres for conditioning and testing (MOD)), which is an international standard for this standard.

Specific examples of hydrophilic fibers for constructing the main sheet 11 include, in addition to cotton, fibers such as silk, wool, hemp, rayon, and cupra (long fibers and short fibers). On the other hand, specific examples of the hydrophobic fibers for constructing the secondary sheet 12 include, in addition to polylactic acid, fibers such as vinylon, nylon, vinylidene, polyester, acrylic, polyethylene, polypropylene, and PET (long fibers and short fibers). Among them, in the present invention, it is preferable to use a nonwoven fabric made of rayon fibers or cotton fibers as the main sheet 11 and a nonwoven fabric made of PET fibers or polylactic acid fibers as the secondary sheet 12.

In the present invention, the present-invention laminated sheet 1 is preferably one in which the weight per unit area of the secondary sheet 12 is larger than the weight per unit area of the main sheet 11. The weight per unit area of the main sheet 11 is preferably 10 to 50 g/m² (more preferably 25 to 35 g/m²), and the weight per unit area of the secondary sheet 12 is preferably 15 to 60 g/m² (more preferably 20 to 40 g/m²).

Further, in the present embodiment, in constructing the present-invention laminated sheet 1, the main sheet 11 and the secondary sheet 12 are laminated by entwining of fibers using a jet water stream. However, the measures for laminating the main sheet 11 and the secondary sheet 12 is not particularly limited. The main sheet 11 and the secondary sheet 12 may be laminated by, for example, pressure-sensitive adhesion, adhesion, or heat fusion.

In the present embodiment, the present-invention external patch 10 is constructed by impregnating the support 2 cut into a shape imitating a human face shape with lotion as the chemical solution 3, but the shape of the support 2 and the type of the chemical solution 3 are not particularly limited.

Further, in the present embodiment, a bag material is selected as the container 4 for constructing the present-invention packaging structure 100, but the form of the container 4 and the material constituting the container 4 are not particularly limited as long as it can enclose the present-invention external patch 10.

It should be noted that the present invention can be implemented in various other forms without departing from the spirit or main features thereof. For this reason, the above-described embodiments are merely examples in all respects and should not be interpreted in a limited manner. The scope of the present invention is indicated by the claims, and is not restricted by the text of the specification. Further, all modifications and changes belonging to the equivalent scope of the claims are within the scope of the present invention.

### INDUSTRIAL APPLICABILITY

The present-invention laminated sheet is suitably used as a support for the present-invention external patch enclosed in a folded state in a container.

What is claimed is:

1. A laminated sheet having a laminated structure of two or three layers in which a secondary sheet is laminated on one side or both sides of a main sheet,
wherein the main sheet is a nonwoven fabric made of hydrophilic fibers, and
wherein the secondary sheet is a nonwoven fabric made of hydrophobic fibers.

2. The laminated sheet according to claim 1,
wherein the main sheet is made of hydrophilic fibers with an official regain of 7.0% or more, and
wherein the secondary sheet is made of hydrophobic fibers with an official regain of 5.5% or less.

3. The laminated sheet according to claim 1,
wherein the main sheet is made of rayon fibers or cotton fibers, and
wherein the secondary sheet is made of PET fibers or polylactic acid fibers.

4. The laminated sheet according to claim 1,
wherein a weight per unit area of the secondary sheet is larger than a weight per unit area of the main sheet.

5. The laminated sheet according to claim 1,
wherein the main sheet has a weight per unit area of 10 to 50 g/m², and
wherein the secondary sheet has a weight per unit area of 15 to 60 g/m².

6. An external patch used by being applied to a skin surface comprising:
a support including the laminated sheet according to claim 1; and
a chemical solution that impregnates the support.

7. A packaging structure in which the external patch according to claim 6 is packaged in a container,
wherein the support is enclosed in the container together with the chemical solution in a state of being folded.
